(19) United States
(12) Patent Application Publication
(10) Pub. No.: US 2025/0131998 A1
(43) Pub. Date: Apr. 24, 2025

(54) METHODS AND DEVICES FOR A CONFIGURABLE MEDICAL INFORMATION PLATFORM

(71) Applicant: The Regents of the University of Colorado, a body corporate, Denver, CO (US)

(72) Inventors: Nikhil MADHURIPAN, Denver, CO (US); Aakriti PANDITA, Denver, CO (US)

(21) Appl. No.: 18/920,752

(22) Filed: Oct. 18, 2024

Related U.S. Application Data
(60) Provisional application No. 63/545,111, filed on Oct. 20, 2023.

(51) Int. Cl. G16H 15/00; G16H 10/60

(52) U.S. Cl. CPC G16H 15/00; G16H 10/60

(57) ABSTRACT

A method and apparatus for connecting a first medical data repository to a first node, the first node configured to obtain a set of medical data from the first medical data repository and refine the set of the medical data, refining, based on one or more parameters, the set of medical data to produce a set of refined medical data; and outputting the set of the refined medical data, the set of the refined medical data configured for seamless integration with a second medical data repository.

### CROSS-REFERENCE TO RELATED APPLICATIONS

This application claims benefit of U.S. provisional patent application Ser. No. 63/545,111, filed Oct. 20, 2023, which is herein incorporated by reference.

### BACKGROUND

### Field

Aspects of the present disclosure generally relate to medical informatics. Specifically, the present disclosure provides methods and devices for a configurable medical information platform.

### Description of the Related Art

Medical informatics are broadly implemented for recording keeping, medical billing, and hospital administration. Medical informatics may be useful to medical providers to facilitate both careful patient care and accurate documentation.

Although current techniques for maintaining medical information are based on technological advancements made over many years, resultant informatics platforms may be insufficiently compatible. For example, a system for medical record keeping may have an architecture inconsistent with an architecture provided for physician notetaking. As a result, medical records associated with an instance of physician notetaking may not accurately reflect medical reality. Correcting such inaccuracies may introduce costly inefficiencies into both the system for medical record keeping and the architecture provided for physician notetaking. Accordingly, there is an impetus to improve the incongruence inherent to medical informatics, including, for example: improving integration of independent medical systems, improving accuracy of data shared across medical systems, improving accuracy of data shared with providers, improving the efficiency sharing medical information, enhancing patient care in association with more efficient information systems, reducing duplicative operations associated with integration of independent medical systems, and the like.

Consequently, there exists a need for further improvements in medical informatics to overcome the aforementioned technical challenges and other challenges not mentioned.

### SUMMARY

The methods and devices of the disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure may provide advantages, such as improved informatics efficiency.

The present disclosure generally relates to a method performed by an apparatus. The method may include connecting a first medical data repository to a first node, the first node configured to obtain a set of medical data from the first medical data repository and refine the set of the medical data, refining, based on one or more parameters, the set of medical data to produce a set of refined medical data, and outputting the set of the refined medical data, the set of the refined medical data configured for seamless integration with a second medical data repository

The present disclosure generally relates to an apparatus. The apparatus may include a memory having executable instructions, and a processor configured to execute the executable instructions and cause the apparatus to perform a method. The method may include connecting a first medical data repository to a first node, the first node configured to obtain a set of medical data from the first medical data repository and refine the set of the medical data, refining, based on one or more parameters, the set of medical data to produce a set of refined medical data, and outputting the set of the refined medical data, the set of the refined medical data configured for seamless integration with a second medical data repository

The present disclosure generally relates to an apparatus. The apparatus may include means for performing a method. The method may include connecting a first medical data repository to a first node, the first node configured to obtain a set of medical data from the first medical data repository and refine the set of the medical data, refining, based on one or more parameters, the set of medical data to produce a set of refined medical data, and outputting the set of the refined medical data, the set of the refined medical data configured for seamless integration with a second medical data repository

The present disclosure generally relates to a non-transitory computer-readable medium having executable instructions that, when executed by a processor of an apparatus, cause the apparatus to perform a method. The method may include connecting a first medical data repository to a first node, the first node configured to obtain a set of medical data from the first medical data repository and refine the set of the medical data, refining, based on one or more parameters, the set of medical data to produce a set of refined medical data, and outputting the set of the refined medical data, the set of the refined medical data configured for seamless integration with a second medical data repository

The present disclosure generally relates to a computer program product embodied on a computer-readable storage medium having code for performing a method. The method may include connecting a first medical data repository to a first node, the first node configured to obtain a set of medical data from the first medical data repository and refine the set of the medical data, refining, based on one or more parameters, the set of medical data to produce a set of refined medical data, and outputting the set of the refined medical data, the set of the refined medical data configured for seamless integration with a second medical data repository

To the accomplishment of the foregoing and related ends, the one or more aspects comprise the features hereinafter fully described and particularly pointed out in the claims. The following description and the appended drawings set forth in detail certain illustrative features of the one or more aspects. These features are indicative, however, of but a few of the various ways in which the principles of various aspects may be employed.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, may admit to other equally effective aspects.

FIG. 1 is an example data flow schematic, operating according to certain aspects of the present disclosure.

FIG. 2 illustrates an example workflow construction diagram, implemented according to certain aspects of the present disclosure.

FIG. 3 and FIG. 4 are screenshots illustrating an example user interface, implemented according to certain aspects of the present disclosure.

FIG. 5 illustrates an example artificial intelligence (AI) node architecture, which may be implemented according to certain aspects of the present disclosure.

FIG. 6 is a flow diagram illustrating certain operations by one or more processors, according to certain aspects of the present disclosure.

FIG. 7 illustrates a schematic of a note bar, which may be implemented according to certain aspects of the present disclosure.

FIG. 8 illustrates a non-limiting example of a workflow implemented on methods and devices described herein.

FIG. 9 illustrates a non-limiting example of a workflow implemented on methods and devices described herein.

FIG. 10 is a flow diagram illustrating certain operations by one or more processors, according to certain aspects of the present disclosure.

FIG. 11 is an example device for performing a method.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

In the following, reference is made to aspects of the disclosure. However, it should be understood that the disclosure is not limited to specifically described aspects. Instead, any combination of the following features and elements, whether related to different aspects or not, is contemplated to implement and practice the disclosure. Furthermore, although aspects of the disclosure may achieve advantages over other possible solutions and/or over the prior art, whether or not a particular advantage is achieved by a given aspect is not limiting of the disclosure. Thus, the following aspects, features, embodiments, and advantages are merely illustrative and are not considered elements or limitations of the appended claims except where explicitly recited in a claim(s). Likewise, a reference to “the disclosure” shall not be construed as a generalization of any inventive subject matter disclosed herein and shall not be considered to be an element or limitation of the appended claims except where explicitly recited in a claim(s).

The present disclosure provides methods and devices for a configurable medical information platform. The platform may provide a functional architecture for flexible and robust implementation of a variety of complex workflows for medical informatics.

Accurate documentation is essential for both patient care and insurance billing. Medical informatics systems function to maintain accurate information regarding patients, providers, and hospital administration procedures. However, medical informatics systems are often incompatible with both provider input information and with other medical informatics systems. Accordingly, medical informatics systems may operate inefficiently as a result of duplicative operations arising from incompatibility with other systems.

Ideally, medical information is recorded, saved, and cataloged for future access using appropriate storage devices. A storage device represents a type of computing hardware utilized for the purpose of housing, moving, or accessing data files and objects. These devices have the ability to retain data on a temporary or permanent basis and can be found as components within or outside of a computer, server, or computing device. In one example, an Electronic Medical Records (EMR) and or/Electronic Health Record (EHR) system may utilize such hardware to store and manage patient-related data, medical records, and associated information. Storage devices used in an EHR system may serve as a secure repository for storing patient records, medical histories, test results, treatment plans, and other healthcare-related data in a digital format. Healthcare providers with robust access to the EHR system can access patient records, retrieving the required information from the storage device. In another example, medical billing systems system may utilize storage device hardware to store billing records, invoices, claims, and payment history.

Both EHR systems and medical billing systems are often in communication with other healthcare systems and databases. Storage devices play a role in managing the storage and retrieval of data relevant to systems that may communicate with one another. An issue arises when storage devices used to manage the storage and retrieval of data integrated across multiple systems are incompatible with one another. Incompatibility may result from incongruous hardware or software, operating system differences, protocol mismatch, lack of common formatting, and the like. As a result of system incompatibility, medical informatics systems may contend with poor inter-system communication and data inconsistency. This may lead to erroneous and duplicative operations by storage devices within the systems, as well as energy and resource waste associated with storage device error.

Complex administrative tasks within healthcare are a consistent source of duplicative operations within medical informatics systems. Generally, many administrative tasks associated with medical practice are inefficient. Note writing and documentation necessitates manual oversight and impacts provider productivity. Maintaining EHR systems may necessitate manual search of EHR systems and may compel providers to enter data that duplicates earlier note-taking. Similarly, to best work with complex, annually-changing algorithms implemented in insurance billing administration, providers may implement time-consuming, error-prone billing processes. Billing processes may duplicate work performed during notetaking and EHR system maintenance. This problem may be compounded for storage devices operating within an EHR system, where input data may be duplicative and difficult to identify and remove, or otherwise incompatible as a result of data type or formatting. Because each of these administrative tasks may be insufficiently complimentary to one another, and because they may produce information that presents a burden to EHR computational systems, both providers and they systems providers utilize may bear the inefficient burden of balancing incongruent and time-consuming administrative tasks.

Currently, there is no viable way to streamline a provider workflow in a manner that reduces wasteful, duplicative tasks. Current healthcare system procedures often adopt a one-size-fits-all approach, which does not address the needs of specific computational systems, individual physicians, and unique specialties.

Aspects of the present disclosure aim to improve the efficiency and accuracy of medical information production, management, and integration with existing EHR and insurance billing systems. The disclosure provides a robust platform architecture that allows providers to generate and customize node-based workflows. Workflows may be enhanced using deterministic logic functionality, and integrated artificial intelligence (AI) functionality, ultimately providing a customizable and user-friendly solution for generating and sharing medical information (e.g., notes, reports, and orders) that mitigates duplicative provider work within a complex and multi-tiered medical system.

Aspects of the present disclosure provide methods and devices that implement a dynamic User Interface (UI) that simplifies individual device interaction with EMR and insurance billing. For example, the device and method may apply a dynamic UI to combine node-based workflows with deterministic logic and advanced AI capabilities that facilitate advantageous note drafting and actions execution that may overcome the limitations of existing EMR and insurance billing systems. By allowing users to build custom workflows, the method and device described herein may improve efficiency and accuracy in medical informatics. Aspects described herein may implement a modular architecture. Ultimately, aspects herein may improve patient care, hospital revenue generation, and healthcare provider productivity.

In some cases, the dynamic UI implemented according to certain aspects may be constructed using a frontend framework. The front end framework may facilitate the construction of interactive and responsive UI component, such as draggable node elements, modals, and panels. Each UI component may be flexibly configured to interact with unique administrative systems within a healthcare system. Additionally, user experience (UX) implemented according to certain aspects may be constructed using a frontend framework. The front end framework may facilitate the construction of interactive and responsive UI component, such as draggable node elements, modals, and panels. Each UI component may be flexibly configured to interact with unique administrative systems within a healthcare system. In some non-limiting example, frontend frameworks like React, React Flow, Angular, or Vue.js may be used in relation to dynamic UI and UX.

In some cases, server-side logic implemented according to certain aspects of the present disclosure may be constructed using a backend framework. For example, the server-side logic may be flexibly configured to enable seamless communication with EHR systems, medical billing systems, AI services, and other external data sources. Communication between devices provided herein and external systems such as EHR may be wired communication (e.g., ethernet, fiber optic communication, and coaxial cable communication), wireless communication (e.g., Wi-Fi, Bluetooth, cellular communication, near field communication (NFC), satellite communication), peer-to-peer (P2P) communication (e.g., P2P, blockchain), client-server communication, voice over internet protocol (VOIP), application programming interface (API) communication, internet of things (IoT) communication, point-to-point communication, and multipoint communication. In some non-limiting examples, the server-side logic may be implemented using backend technologies such as Node.js, Django, Flask, or Ruby on Rails.

In some cases, database management systems may be implemented to store and manage user-generated workflows, custom nodes, and other relevant data. In some non-limiting examples, database management systems may be implemented using MySQL, PostgreSQL, or MongoDB to store and manage user-generated workflows, custom nodes, and other relevant data.

In some cases, advanced AI capabilities may be implemented to provide natural language processing, summarization, and information extraction capabilities. In some non-limiting examples, advanced AI capabilities may be implemented as part of an AI node using OpenAI's GPT series models (GPT-3.5, GPT-4, GPT-5), Anthropic's Claude series models, or other large language models, including models fine tuned to domain specific tasks, to provide natural language processing, summarization, and information extraction capabilities. These models can be accessed through APIs or locally hosted on the server.

In some cases, data visualization systems may be implemented to generate interactive and dynamic visualizations (e.g., plots, charts) based on the input data. In some non-limiting examples, the data visualization systems may implement data visualization libraries like D3.js, Chart.js, or Seaborn (Python).

In some cases, workflow management systems may be implemented to enable users to create, edit, and manage custom workflows comprising various node elements. This may include support for drag-and-drop functionality, node connections, and data flow management. In some non-limiting examples, the workflow management system may implement node based workflow creation tools like React-flow, Rete.js, and other node and diagramming libraries.

According to aspects of the present disclosure, the methods and devices described herein may be implemented according to a processing system architecture comprising at least one or more central processing units (CPUs) operating alone or in combination with one or more graphics processing units (GPUs). The one or more CPUs and/or the one or more GPUs may perform the procedures according to a non-transitory computer readable medium that causes the one or more CPUs and/or the one or more GPUs to perform any and all steps of the procedure. Each of the one or more CPUs may be utilized in combination with a memory having the computer readable medium stored thereon. Each of the one or more CPUs be utilized in combination with one or more processors. Each of the one or more processors may be parallel processors or processors operating in sequence. Each of the one or more GPUs may be utilized in combination with a memory having the computer readable medium stored thereon. Each of the one or more GPUs be utilized in combination with one or more processors. Each of the one or more processors may be parallel processors or processors operating in sequence. Each of the CPUs and the GPUs may operate independently, or may operate using a message passing interface (MPI) enabling communication between one or more parallel processors for performing the procedure. This may include CPU-CPU communication, CPU-GPU communication, and/or GPU-GPU communication.

As used herein, the term “data” and the term “data type” encompass a wide variety of types of data. For example, “data” and/or “data type” may include integers, floating-point numbers, characters, strings, booleans, arrays, lists, tuples, dictionaries, sets, date and time values, complex numbers, decimals, binary data, null or None values, enumerations, pointers, custom objects and classes, files, JSON (JavaScript Object Notation), XML (extensible Markup Language), UUIDs (Universally Unique Identifiers), IPv4 and IPV6 addresses, long integers, short integers, big integers, character arrays, binary large objects, matrices, time intervals, rational numbers, graphs, spatial data types, regular expressions, monetary data types, queues, stacks, hash tables, bitsets, color representations, sound and audio data types, temperature measurements, units of measurement, scientific notation, data frames, neural networks, tensors, embeddings, graph neural networks (GNNs), time series data types, probabilistic data types, N-dimensional arrays, semantic graphs and various data structures used for deep learning models, such as arge language models and vision language models, such as Open Nueral Network Exchange (ONNX), Pytorch .pth/.pt files, and HuggingFace Safetensors. “Data” and/or “data type” may also refer to a data representation form not listed herein, but understood by one having ordinary skill in the art as “data” or a “data type.” Any presentation of specific “data” or “data types” incorporated herein is an example presented to facilitate understanding and is not limiting of the scope, applicability, or aspects set forth in the disclosure.

### Example Node-Based Workflow

According to certain aspects of the present disclosure, a functional architecture implemented for flexible and robust implementation of a variety of complex workflows may be accessible to a user via a series of nodes and node-based medical informatics workflows. The medical informatics workflows may be customized by a user via UI according to one or more nodes. In one example, the nodes may be operable in sequence with one another, such that output from a first node may provide input for another node. In one example, the nodes may be operable in parallel with one another, such that two or more nodes may operate near or at the same time to produce a target output. In one example, the nodes may be operable independent with one another, such that any of the nodes within the platform may operate autonomously.

According to certain aspects of the present disclosure, the nodes are configurable via a UI. In one example, a first node may be placed adjacent to a second node on a UI display, such as to the left of the first node, to the right of the first node, above the first node, below the first node, or diagonal to the first node. In one example, a first node may be placed adjacent to a set of nodes on a UI display. In one example, a first node may not be adjacent to a second node or a set of nodes on a UI, but may be logically linked within a workflow. Logical links between nodes may be utilized for a certain workflow, and may be configurable to achieve desired results. Logical links may be visually displayed via the UI, or otherwise incorporated into the UX.

In a first example, the platform may provide a reasoning node to a user, to be optionally included as part of a customizable medical informatics workflow. The reasoning node may be optionally implemented to perform any arithmetic operation in response to an input. The arithmetic operation may be performed as an operation using all or some portion of an input. Arithmetic operation may include addition, subtraction, multiplication, division, modulo, exponentiation, or any other suitable arithmetic operation. The reasoning node may be optionally implemented to perform any comparison operation. The comparison operation may be implemented between any input and any other datum, between a first portion of an input and a second portion of an input, or any other suitable comparison operation. In some cases, the reasoning node may output a result. In certain non-limiting examples, output from the reasoning node may be in a python code format (e.g., JSON, dict, array). In some cases, the reasoning node may be available to a user in a note editor within a UI. The reasoning node may be movable within a UI, and may accept input from a user, from an external database, from an internal database, or from another node in the platform.

In a second example, the platform may provide a programming node to a user, to be optionally included as part of a customizable medical informatics workflow. The programming node may be optionally implemented to perform any complex logical operation. Complex logical operation may include complex logic information provided by the user (e.g., via direct input). The programming node may have input connectors capable of obtaining and assigning inputs based on the complex logic implemented in the programming node. In one example, the input connectors may process data including numeric data, sequence type data, Boolean data, set data, dictionary data, and binary type data. The input connector may connect to a UI, an external database, an internal database, or another node in the platform. The programming node may have output connectors capable of outputting output information based on the complex logic implemented in the programming node. In one example, the output connectors may process data including numeric data, sequence type data, Boolean data, set data, dictionary data, and binary type data. The output connector may connect to a UI, an external database, an internal database, or another node in the platform. Input and output connectors may allow a user to implement more versatile and efficient notetaking practices. In one non-limiting example, the programming node may implement complex logical operations using Python. In some cases, the programming node may be available to a user in a note editor within a UI. The programming node may be movable within a UI, and may accept input from a user, from an external database, from an internal database, or from another node in the platform.

In a third example, the platform may provide a visualization node to a user, to be optionally included as part of a customizable medical informatics workflow. The visualization node may be optionally implemented to perform any display capability within the UI. The visualization node may accept input information (e.g., numerical data) and generate visualization based on the input. The visualization may connect to a UI input display, an external database, an internal database, or another node in the platform. In one non-limiting example, the visualization node may be optionally connected to the output connector of the programming node. In one non-limiting example, the visualization node may be equipped to operate using Seaborn, D3.js, Chart.js, and the like to create the desired graphical representations. In one non-limiting example, the visualization node may be equipped to operate using visualization techniques provided by the user. The visualization node may output display information either as a single display item, or as a set of display items. In some cases, the user may select from the one or more display items to best represent the input data. In one non-limiting example, the one or more display item may be presented using a selection of data visualization formats, including charts, tables, JSON, or other array-based representations. In some cases, the visualization node may be available to a user in a note editor within a UI. The visualization node may be movable within a UI, and may accept input from a user, from an external database, from an internal database, or from another node in the platform.

In a fourth example, the platform may provide a text node to a user, to be optionally included as part of a customizable medical informatics workflow. The text node may be optionally implemented to allow rich text formatting and inline editing. The text node may be capable of accepting input information (e.g., text, numerical data, Boolean) and generate text based on the input information. In some cases, generating text based on the input information may be based on based on the logic implemented in the text node. In some cases, the text node may be movable within a UI, and may accept the input information from a user, from an external database, from an internal database, or from another node in the platform. The input information may be fixed or conditional text. The text node may be capable of outputting output information based on the logic implemented in the programming node. In some non-limiting examples, the output information may be redefined and/or custom text. In some cases, the text node may be available to a user in a note editor within a UI.

In a fifth example, the platform may provide a data retrieval node to a user, to be optionally included as part of a customizable medical informatics workflow. The data retrieval node may be capable of retrieving medical information (e.g., patient data, demographic data, vital sign information, order information, lab results, notes). In some cases, the data retrieval node may retrieve medical information from one or more EMR systems. In some cases, the data retrieval node may retrieve medical information from one or more medical billing systems. The data retrieval node may be capable of retrieving data in a manner sufficient to maintain the integrity of the data. The data retrieval node may perform data retrieval based on the logic implemented in the data retrieval node. The data retrieval node may be capable of querying and filtering data based on custom criteria. These custom criteria include all data available through the electronic medical record including but not limited to date and time, numerical range, data source, data type with multiple levels of specificity such as a specific lab value, imaging report of a specified body part, note written by physicians or other healthcare providers, demographics, diagnosis codes, encounter types, medication types, and medication administration records. The custom criteria may be implemented at the data retrieval node according to user input, or internal logic. In some cases, querying data may include sending a request (e.g., via a select query) for information to an EMR system, and electronic health record (EHR) system, a medical billing system, a local database, or any other relevant data source. The data retrieval node may query data using syntax defined within the platform, or accessed from an external source. Filtering data may include processing retrieved data to produce a subset of the retrieved data. In some cases, filtering data may be optional. Filtering may be achieved via auto-filtering procedures, advanced filtering procedures, or any other filtering procedures capable of producing a desired output. The data retrieval node may be capable of outputting processed or unprocessed data. The data retrieval node may produce output data based on the logic implemented in the programming node. Processed data may be data retrieved by the data retrieval node and made subject to certain operations or analysis techniques. Unprocessed data may be raw data retrieved by the data retrieval node. In some cases, the data retrieval node may be available to a user in a note editor within a UI. The data retrieval node may be movable within a UI, and may accept input from a user, from an external database, from an internal database, or from another node in the platform.

In a sixth example, the platform may provide an order node to a user, to be optionally included as part of a customizable medical informatics workflow. The order node may be capable of constructing and managing medical orders (e.g., prescriptions, lab tests, or imaging studies, and the like). The order node may construct and manage medical orders based on the logic implemented in the data retrieval node. In one example, the order node may construct and manage medical orders by extracting medical information, user directives, and the like from a source (e.g., a user input, an external source, another node). After extracting, the order node may interpret extracted data and produce a medical order according to a format. The format may be pre-defined within the node, provided from an external data source, or configured by the user. The order node may send the medical order to a desired reception point, which may include another node, an internal database, an external database, or an external provider. The order node may store the medical order in a catalog, which may be made available for later reference. In some cases, the order node may be integrated with an EHR, EMR, or medical billing system. Integration may facilitate seamless and efficient order management. In some cases, the order node may automatically populate order details based on certain information, such as patient data, provider data, and the like. In some cases, the order node may be available to a user in a note editor within a UI. The order node may be movable within a UI, and may accept input from a user, from an external database, from an internal database, or from another node in the platform.

In a seventh example, the platform may provide a task node to a user, to be optionally included as part of a customizable medical informatics workflow. The task node may be capable of constructing, assigning, and tracking tasks. The task node may construct, assign, and track tasks based on the logic implemented in the task node. In some cases, tasks may be related to patient care (e.g., follow-up appointments, care coordination, patient education, and the like). In one example, the task node may construct tasks by extracting medical information, user directives, and the like from a source (e.g., a user input, an external source, another node). After extracting, the task node may interpret extracted data and produce a task according to a format. The format may be pre-defined within the node, provided from an external data source, or configured by the user. The task node may send the task to a desired reception point, which may include another node, an internal database, an external database, or an external provider. In some cases, sending the task may include assigning the task (e.g., to the recipient). The task node may store the task in a catalog, which may be capable of tracking a set of tasks. Tracking tasks may include monitoring a qualities of the task, such as due date, status information, priority information, provider information, patient information, location information, completion information, and the like. In some cases, tracking may include sending reminders to a recipient about a task. Reminder format and frequency may be pre-defined within the node, provided from an external data source, or configured by the user. In some cases, the task node may be integrated with an EHR, EMR, or medical billing system. Specifically, the task node may be integrated with a system's task management module or external task management tools. The task node may be capable of displaying a summary of open tasks within the UI. The task node may display tasks based on the logic implemented in the task node. The task node may allow a user to mark tasks as completed or update information associated with a task. In some cases, the task node may be available to a user in a note editor within a UI. The task node may be movable within a UI, and may accept input from a user, from an external database, from an internal database, or from another node in the platform.

In an eighth example, the platform may provide an artificial intelligence (AI) node to a user, to be optionally included as part of a customizable medical informatics workflow. The AI node may be capable of various tasks, each of which may be performed based on the logic implemented in the AI node. Logic implemented in the AI node is highly customizable, and may be pre-defined within the node, provided from an external data source, or configured by the user. In some cases, the AI node may utilize a server based external AI provider to provide an inferential model. In some cases, the AI node may utilize a locally hosted AI model to provide an inferential model. In one non-limiting example, the AI node may be capable of summarizing lengthy text-based information, such as medical texts, patient information and the like. In one non-limiting example, the AI node may be capable of converting prose into easy-to-read bulleted lists. In one non-limiting example, the AI node may be capable of drafting discharge summaries based on the patient's medical history, treatment received, and follow-up recommendations. In one non-limiting example, the AI node may be capable of constructing concise medical histories for patients with multiple conditions. In one non-limiting example, the AI node may be capable of identifying and extracting key information from lab reports or imaging studies. In one non-limiting example, the AI node may be capable of drafting referral letters to specialists based on a patient's diagnosis and treatment plan. In one non-limiting example, the AI node may be capable of summarizing a patient's progress over time, including improvements or setbacks in their condition. In one non-limiting example, the AI node may be capable of identifying potential gaps or inconsistencies in provided data, in care or areas for improvement based on the patient's medical history and treatment plan. In one non-limiting example, the AI node may be capable of checking the note for clinical documentation integrity and level of billing. In some cases, the AI node may be available to a user in a note editor within a UI. The AI node may be movable within a UI, and may accept input from a user, from an external database, from an internal database, or from another node in the platform.

In a ninth example, the platform may provide note bar to a user, to be optionally included as part of a customizable medical informatics workflow. The note bar may be a vertical bar on the side of a note editor page (e.g., the right side of the note editor page). In some cases, any node described herein, either alone or in combination, may be fed into the note bar to generate a final output. The final output may be comprehensive medical document, termed a “final note”. The final note may be produced based on a given workflow formed by the nodes described herein. A given workflow for producing the final note may involve a several-step manipulation of medical informatics relevant to patient information in EHR systems, EMR systems, medical billing systems, and the like. A given workflow for producing the final note may involve proactive monitoring, analysis, and notification steps performed by a device implementing the platform.

### Aspects Related to Customizable Medical Documentation

According to certain aspects of the present disclosure, flexible and robust implementation of customizable medical informatics workflows may be facilitated by implementation of a functional architecture. The functional architecture, which is implemented within a workflow platform, may enable precise workflow management by providing users with the ability to adjust customizable nodes within a workflow while mitigating potential data errors that may be inherent to customization. Additionally, the functional architecture may support seamless integration of medical information systems. Seamless integration may reduce duplicative data management operations, reducing system inefficiency and conserving system power and resources.

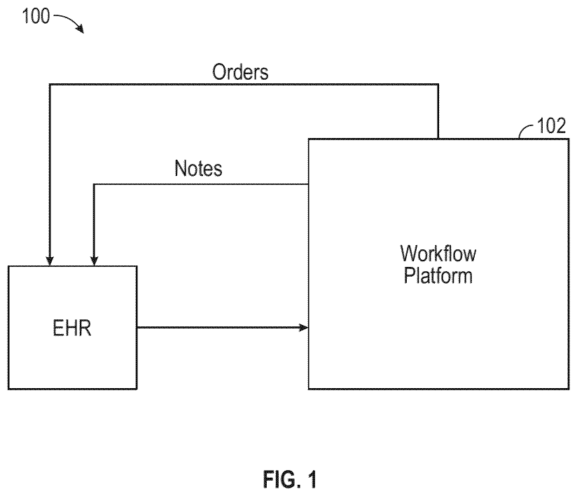

FIG. 1 is an example data flow schematic illustrating the path of medical informatics within the platform, according to certain aspects of the present disclosure. The workflow platform 102 may be customized using one or more nodes, such as nodes described above. The workflow platform may be implemented via one or more processors. After activation, the processors output notes and orders to an EHR system 104, and the EHR system outputs data, sending it to the processors. In some cases, the processor outputs notes and orders using at least a text node and an order node. In some cases, the EHR system may output data in response to a query from a data retrieval node within the processors. The EHR system may output data and send the data to the processors in response to obtaining notes and orders from the processors. The EHR system may output data and send the data to the processors independent from obtaining notes and orders from the processors.

In some cases, the one or more processors implemented in accordance with the workflow platform may send other types of data instead of or in addition to notes and orders. Other types of data may include tasks, numerical values, reminders, and the like. In some cases, the EHR system of FIG. 1 may be an EMR system, a medical billing system, or any other system with which the processors may communicate.

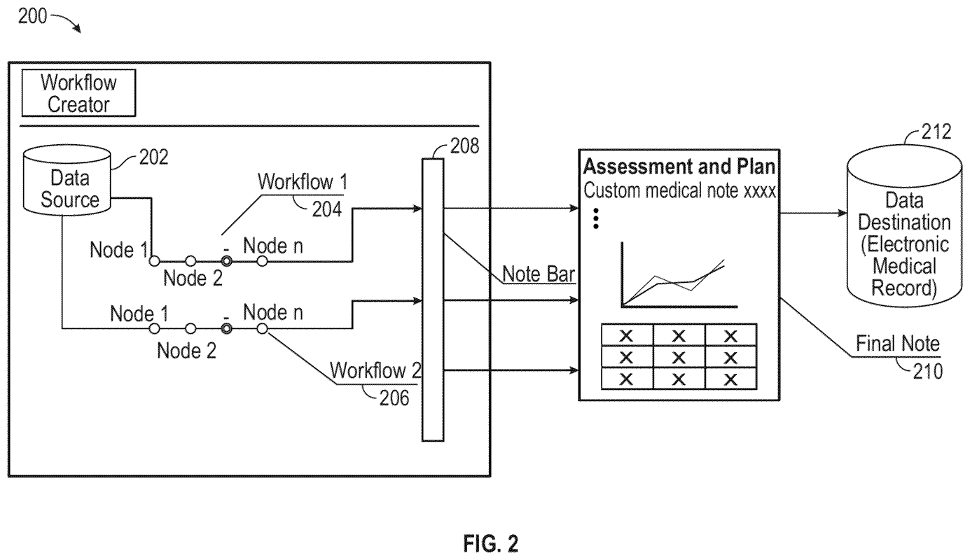

FIG. 2 illustrates an example workflow construction architecture 200, which may be implemented as part of a UI of the workflow platform. The architecture 200 includes a data source 202, a first workflow 204, a second workflow 206, a note bar 208, a final note panel 210, and a data sink 212. The data source 202 is linked to the first workflow 204 and the second workflow 206. The first workflow 204 and the second workflow 206 may have a set number of nodes (e.g., node 1, node 2, . . . , node n), which may be understood with reference to the description of nodes, above. The workflows 204, 206 are linked to a note bar 208. The note bar outputs a final note 210. In some cases, the final note may display custom medical note text, which may include visualization and values selected based on first workflow 204 and the second workflow 206. The final note may be output to a data sink 212 and stored for categorization and further use. In some cases, the data sink may be an EHR system, an EMR system, a medical billing system, an internal data storage system, and the like. The UI may be intuitive, user-friendly, and visually appealing, ensuring that providers can customize and manage workflows for generating medical notes, reports, orders, and the like.

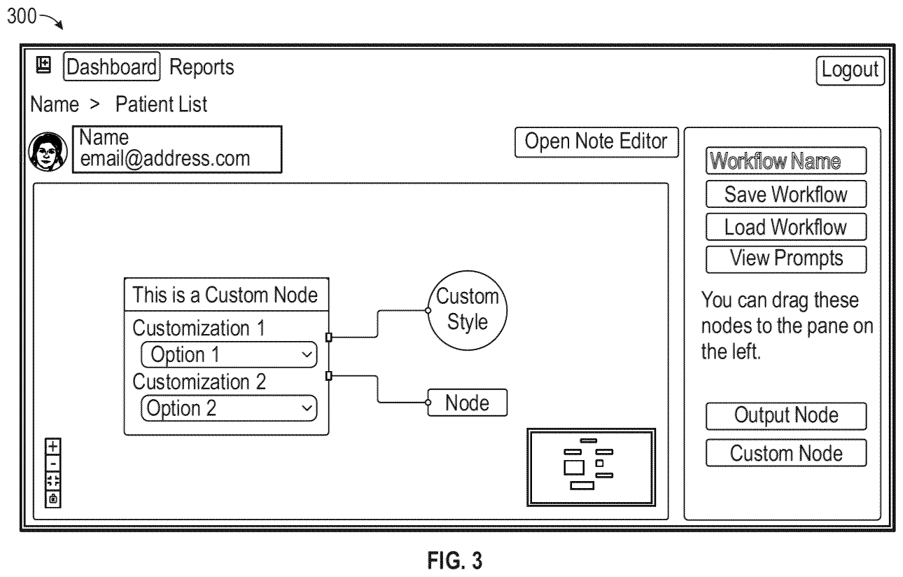

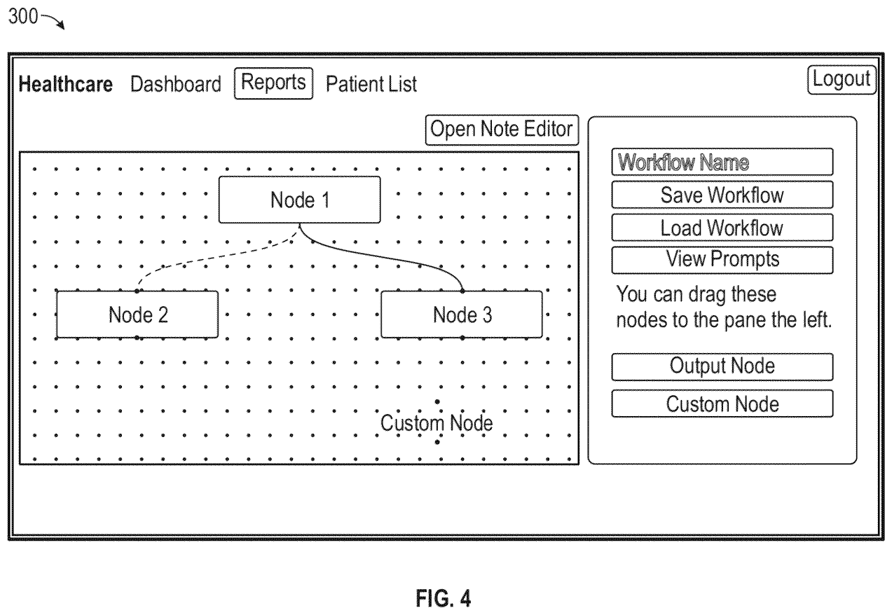

FIG. 3 and FIG. 4 are screenshots illustrating an example user interface 300 for constructing a workflow using the one or more processors implemented in association with the workflow platform. As illustrated, each node element may be represented by a draggable card with a distinct icon, label, and color scheme to differentiate between node types and functionalities. Draggable cards can be dragged and dropped onto a canvas area in the note editor, allowing users to visually arrange and connect the nodes to create custom workflows, according to aspects of the present disclosure. To establish connections between nodes, each card may features input and output connection points, represented by small, circular icons on the edges of the card. Users can click and drag from an output connection point of one node to an input connection point of another node, creating a visual link between them. This link may be associated with the flow of data or logic between the connected nodes, as described in detail above. Each connection point may have labels or hover text describing the accepted input type for the target node, as well as type of output for the target node.

In some cases, when a node card is clicked, a properties panel or modal window may appear, providing users with options to configure a node's specific functionality, settings, and data sources (which may be further understood with reference to the discussion of example nodes, above). This panel or modal window may include dropdown menus, checkboxes, text input fields, and other UI components that may be suitable for customization. Additionally, a help tooltip or information icon may be present on each node card, offering brief explanations of the node's functionality and capabilities. Hovering over or clicking this icon displays the tooltip, which may also include a “Learn More” link directing users to a more detailed help article or tutorial about the node and its functionalities. The canvas area in the note editor features a grid layout, snap-to-grid functionality, and zoom controls, allowing users to organize and position nodes with precision and ease. A “Save Workflow” button may be provided to save the custom workflow for future use or modifications. The node-based workflow UI aims to facilitate efficient, customizable, and user-friendly medical documentation, utilizing both deterministic logic and large language model capabilities.

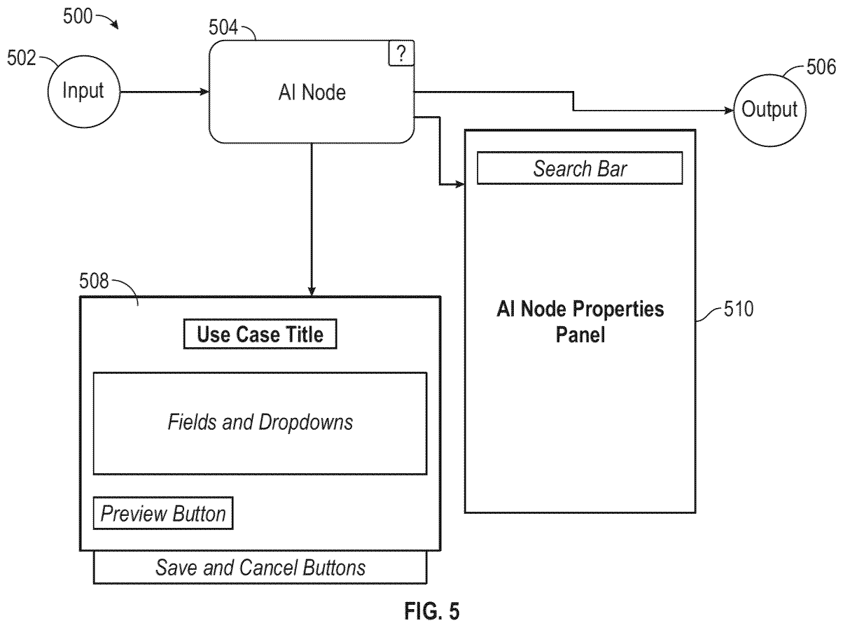

FIG. 5 illustrates an example AI node architecture 500, which may be implemented, via one or more processors, as part of a UI of the workflow platform. The architecture includes an input 502, an AI node 504, and an output 506. The AI node 504 may display at least one of a selection panel 508 and a node property panel 510 that may be accessible to a user at a UI. As discussed above, the AI node may be capable of various tasks, each of which may be performed based on the logic implemented in the AI node. Logic implemented in the AI node is highly customizable, and may be pre-defined within the node, provided from an external data source, or configured by the user. In some cases, the AI node may utilize a server based external AI provider to provide an inferential model. The AI node may utilize a locally hosted AI model to provide an inferential model

The input 502 may take input data from a source. The source may be another node provided by the one or more processors implemented in association with the workflow platform, another internal source, an external source (e.g., an EMR system, an EHR system, a medical billing system), a pre-configured source, and the like. Data may be any data type compatible with the internal logic of the AI node 504. Non-limiting examples of data types include numeric data, sequence type data, Boolean data, set data, dictionary data, binary type data, image data, and the like. Input data may be utilized by the AI node 504, and processed according to customizable AI node properties.

The AI node 504 may be monitored and customized in a manner advantageous to reduce duplicative operations. Customization may be pre-configured in the one or more processors implemented in association with the workflow platform, configured based on an external platform (e.g., an EMR system, an EHR system, a medical billing system), configured by a user, or otherwise configured to achieve a suitable efficiency target. In some cases, the AI node may be customized using the selection panel 508 and the node property panel 510. The selection panel may include a set of category fields and drop-down items, which may be substantially center on the selection panel. The selection panel may be a draggable card including a preview button, a save button, and a cancel button, which may be optionally displayed below the set of category fields and drop-down items. The node property panel may be a draggable card containing the AI Node title and an icon representing an AI engine. The node property panel may use different icons or labels to represent various AI functionalities/available models. Clicking on the card can expand the AI Node Properties to facilitate ease of customization. In one example, a panel may slide out or pops up when the AI Node card is clicked. The node property panel may also contain a search bar at the top of the panel to allow users to quickly find and enable specific use cases, which are discussed in detail above. In some cases, the node property panel may include a list of AI use cases with toggle switches to enable and/or disable specific functionalities. Each use case may have a short description and an optional settings icon for additional configuration. In some cases, when a settings icon is clicked, a modal window may open. The modal window may allow users to customize the AI functionality for a specific use case, such as selecting specific data sources, adjusting parameters, or choosing output format. In some cases, the node property panel may provide clear guidance and examples for each configuration parameter captured within the AI node. Configuration parameters may include, but are not limited to, prompt methods such as prompt string, chain of thought, agents model hyperparameters including temperature, Top P, Top K, maximum length, stop sequences, frequency penalty, repetition penalty, presence penalty, and any other suitable configuration parameter not listed herein but deemed suitable for use by those having ordinary skill in the art. The node property panel may also offer a preview of the AI functionality output based on the selected configuration settings.

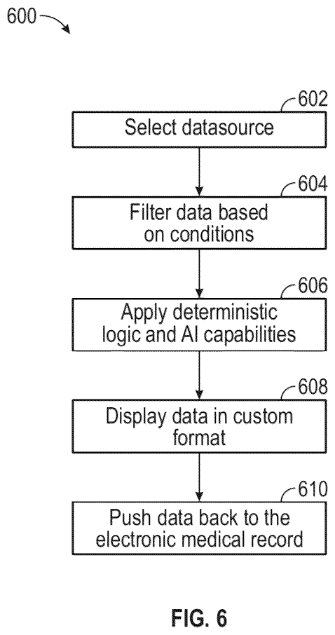

FIG. 6 a flow diagram illustrating certain operations 600 by one or more processors implemented in association with the workflow platform. In some cases, operations 600 are implemented within the AI node architecture 500 of FIG. 5. At operation 602, the processors select an input data source. Data source selection may be pre-configured within the workflow platform, customized by a user, or assigned according to external protocols. At operation 604, the processors filter data based on certain conditions. Certain conditions may include data type and subtype. For example, data type and subtype may include a type clinical note of subtype speech therapy within a time/date range, or imaging data of subtype computed tomography of the abdomen for duration of current hospital admission. At operation 606, the processors apply deterministic logic and AI capabilities to the filtered data. The deterministic logic and AI capabilities may be pre-configured within the workflow platform, customized by a user, or assigned according to external protocols. At operation 608, the processors display the data according to a format. The format may be pre-configured within the workflow platform, customized by a user, or assigned according to external protocols. At operation 610, the processors send data to an external medical system. The data sent to the external medical system may be tailored by the processors implementing the workflow platform for support seamless integration with the external medical system, reducing duplicative operations at the external medical system and conserving associated power and storage resources. The external medical system may include an EHR system, and EMR system, a medical billing system, and the like.

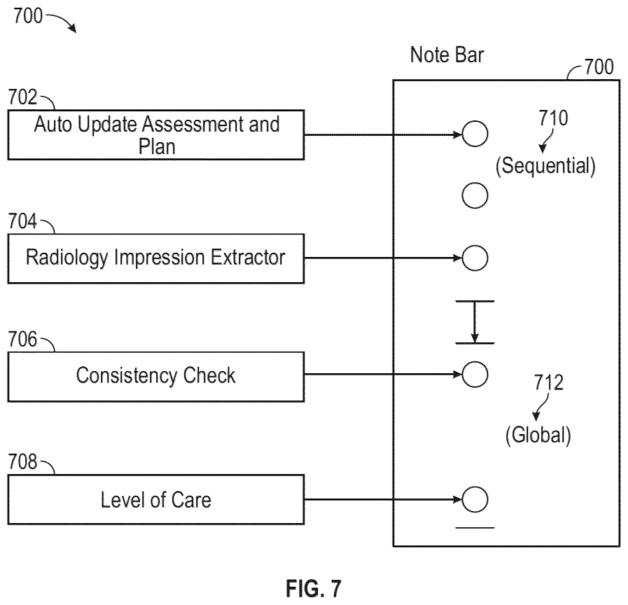

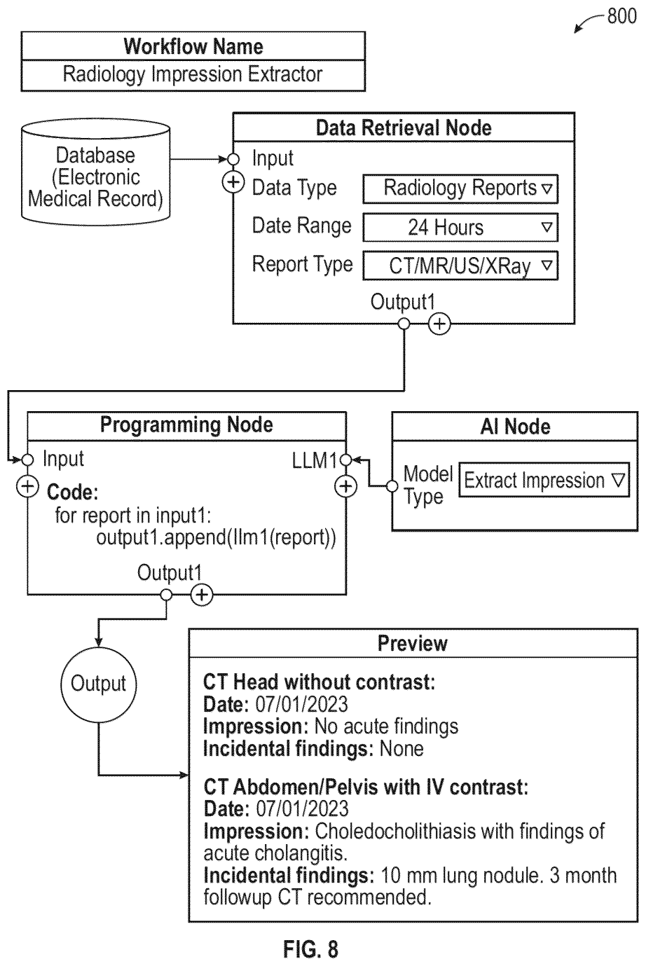

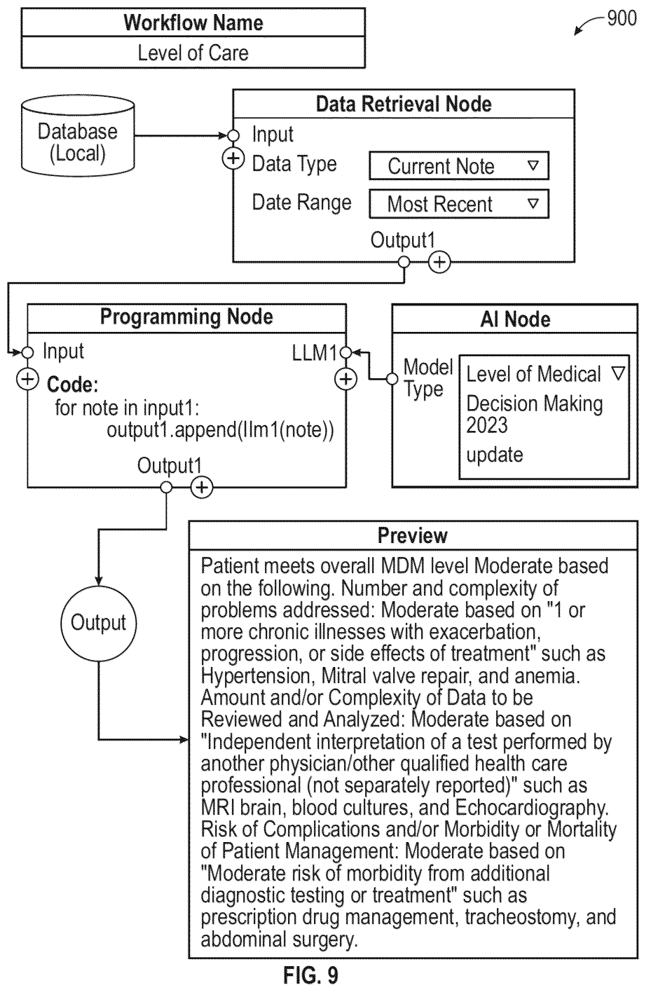

FIG. 7 illustrates a schematic of a note bar 700. According to certain aspects of the present disclosure, the AI node architecture 500 may be linked to the note bar 700 within the workflow platform UI. The note bar may include a presentation of sequentially linked nodes 710 and a presentation of global nodes 712. Sequentially linked nodes may be node with linked inputs and outputs. Global nodes may be nodes processing data independent of the sequentially linked nodes. Non-limiting examples of sequentially linked nodes include a node 702 to auto-update an assessment and plan, a node 704 to extract a radiology impression from input data from node 702, and a node 706 for validating output from node 704. The node 704 to extract a radiology impression may be further understood with reference to FIG. 8. A non-limiting example of a global node includes a level of care node 708 that may accept input information from any source described above. The level of care node 708 may be further understood with reference to FIG. 9.

In some cases, the note bar 700 may present a preview of AI functionality output implemented within a given workflow based on the current configuration settings. The note bar may also provide clear guidance and examples for any given configuration parameter. Configuration parameters may include data type, timing information, integration protocol, compliance protocol, scalability information, automation information, link information, and the like. More specifically, configurations parameters may include a data type, a data filter, a data source, conditional logic, programming logic, an arithmetic operator, a comparison operator, a custom text input, a text formatting, an input connector, an output connector, a data visualization format, an AI model, and parameters to adjust performance the AI model.

Aspects of the present disclosure may allow users to construct flexible, robust medical informatics workflows. The architecture provided by methods and devices provided herein functions to mitigate erroneous operations inherent both to customization of a workflow and to data flow between medical communication systems. By implementing methods and devices disclosed herein, a user may be able to reduce duplicative data management operations, reducing system inefficiency, reduce personal inefficiency, and conserve system power and resources.

### Example Workflows

FIG. 8 illustrates a non-limiting example of a workflow 800 implemented on methods and devices described herein. The workflow may be constructed via a UI. The UI may displayed on a device or system having a display. At the top-left of the display, the workflow is titled within a “workflow name” box. Here, the workflow is titled “radiology impression extractor.” The workflow links a data source, here an EMR database, to a data retrieval node, such that the data sources transfers input (e.g., “Input1”) for the data retrieval node. The data retrieval node is configured to retrieve and refine information from the data source. Accordingly, the data retrieval node is configured to be compatible with the data source. In the example provided, the data retrieval node is at least partially configured by a user via a set of drop down menu options. The drop down menu options may be pre-configured within the workflow platform, customized by a user, or assigned according to external protocols. Here, the user selects “radiology reports” from a “Data type” drop down box, selects “24 hours” from a “Date range” drop down box, and selects “CT/MR/US/XRay” from a “Report type” drop down box. The data retrieval node then outputs a set of refined data (e.g., “Output1”) based on the configuration of the data retrieval node.

The set of refined data becomes input (e.g., “Input1”) for a programming node. The programming node is configured to process from the data retrieval node. Accordingly, the programming node is configured to be compatible with the data retrieval node. Additionally, the programming node is configured to process data from the AI node. Accordingly, the programming node is configured to be compatible with the AI node. In the example provided, the AI node transfers an input (e.g., a language learning model (LLM), “LLM1”) for the programming node to be utilized alongside input from the data retrieval node. The AI node is at least partially configured by a user via a set of drop down menu options. The drop down menu options may be pre-configured within the workflow platform, customized by a user, or assigned according to external protocols. Here, the user selects “Extract impression” from a “Model type” drop down box. The AI node then outputs a set of refined data (e.g., “LLM1”) based on the configuration of the AI node. In the example provided, the programming node is at least partially configured to extract a “report” object from Input1, apply LLM1 obtained from the AI node to the report object, and output the results (e.g., “Output1”) of the application of the LLM to the report object. The programming node may be pre-configured within the workflow platform, customized by a user, or assigned according to external protocol. A user may preview Output 1 a preview window. Output1, which may be optimized for integration with external systems, may be later sent to another system, such as a EMR system, a EHR system, or a medical billing system.

FIG. 9 illustrates a non-limiting example of a workflow 900 implemented on methods and devices described herein. The workflow may be constructed via a UI. The UI may displayed on a device or system having a display. The workflow is titled within a “workflow name” box. Here, the workflow is titled “level of care.” The workflow links a data source, here an internal database, to a data retrieval node, such that the data sources provides input (e.g., “Input1”) for the data retrieval node. The data retrieval node is configured to retrieve and refine information from the data source. Accordingly, the data retrieval node is configured to be compatible with the data source. In the example provided, the data retrieval node is at least partially configured by a user via a set of drop down menu options. The drop down menu options may be pre-configured within the workflow platform, customized by a user, or assigned according to external protocols. Here, the user selects “Current note” from a “Data type” drop down box, and selects “most recent” from a “Date range” drop down box. The data retrieval node then outputs a set of refined data (e.g., “Output1”) based on the configuration of the data retrieval node.

The set of refined data becomes input (e.g., “Input1”) for a programming node. The programming node is configured to process from the data retrieval node. Accordingly, the programming node is configured to be compatible with the data retrieval node. Additionally, the programming node is configured to process data from the AI node. Accordingly, the programming node is configured to be compatible with the AI node. In the example provided, the AI node provides an input (e.g., a language learning model (LLM), “LLM1”) for the programming node to be utilized alongside input from the data retrieval node. The AI node is at least partially configured by a user via a set of drop down menu options. The drop down menu options may be pre-configured within the workflow platform, customized by a user, or assigned according to external protocols. Here, the user selects “Level of Medical Decision Making 2023 update” from a “Model type” drop down box. The AI node then outputs a set of refined data (e.g., “LLM1”) based on the configuration of the AI node. In the example provided, the programming node is at least partially configured to extract a “report” object from Input1, apply LLM1 obtained from the AI node to the report object, and output the results (e.g., “Output1”) of the application of the LLM to the report object. The programming node may be pre-configured within the workflow platform, customized by a user, or assigned according to external protocol. A user may preview Output 1 a preview window. Output1, which may be optimized for integration with external systems, may be later sent to another system, such as a EMR system, a EHR system, or a medical billing system.

### Example Method

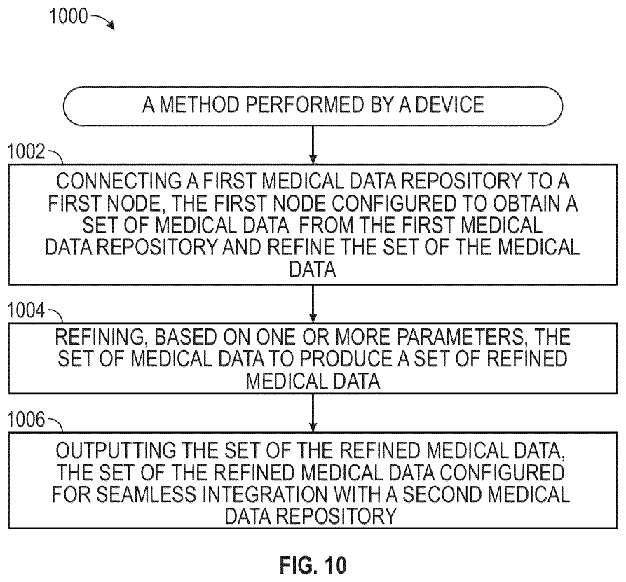

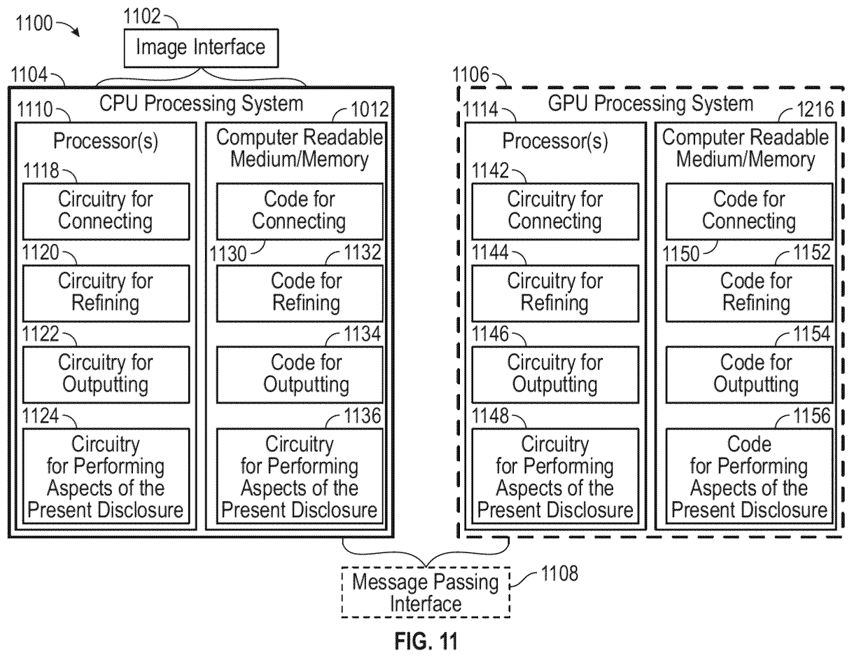

FIG. 10 depicts a method 1000 performed by a device. The device may implement one or more CPUs, such as the CPUs of the device 1100 of FIG. 11.

Method 1000 begins at 1002 with an apparatus connecting a first medical data repository to a first node, the first node configured to obtain a set of medical data from the first medical data repository and refine the set of the medical data.

Method 1000 continues to step 1004, with an apparatus refining, based on one or more parameters, the set of medical data to produce a set of refined medical data.

Method 1000 continues to step 1006, with an apparatus outputting the set of the refined medical data, the set of the refined medical data configured for seamless integration with a second medical data repository.

In one aspect, method 1000, or any aspect related to it, may be performed by an apparatus, such as device 1100 of FIG. 11, which includes various components operable, configured, or adapted to perform the method 1000. Device 1100 is described below in further detail.

Note that FIG. 10 is just one example of a method, and other methods including fewer, additional, or alternative steps are possible consistent with this disclosure.

### Example Device

FIG. 11 depicts aspects of an example workflow platform device 1100. In some aspects, the device 1100 comprises one or more CPUs, one or more GPUs, or both as described above with respect to FIG. 9.

The device 1100 includes a CPU processing system 1104 coupled to an image interface 1102 (e.g., a user interface or and/or an image generator such as a display screen on a mobile device or desktop). The CPU processing system 1104 may be configured to perform processing functions for the device 1100, including operations 600 of FIG. 6 and method 1000 of FIG. 10 performed by the device 1100.

The CPU processing system 1104 includes one or more processors 1110. The one or more processors 1110 are coupled to a computer-readable medium/memory 1112 via a bus. The one or more processors 1110 and the computer-readable medium/memory 1112 may optionally communicate with the one or more processors 1114 and the computer-readable medium/memory 1116 of the graphics processing unit (GPU) processing system 1106 via a message passing interface (MPI) 1108. In certain aspects, the computer-readable medium/memory 1112 is configured to store instructions (e.g., computer-executable code) that when executed by the one or more processors 1110, cause the one or more processors 1110 to perform the method 1000 described with respect to FIG. 10, or any aspect related to it, and/or the operations 600 of FIG. 6, and any aspects related to them. Note that reference to a processor performing a function of device 1100 may include one or more processors performing that function of device 1100.

In the depicted example, computer-readable medium/memory 1112 stores code (e.g., executable instructions) 1130-1136 for performing techniques described herein, according to aspects of the present disclosure. Processing of the code 1130-1136 may cause the device 1100 to perform the method 1000 described with respect to FIG. 10, or any aspect related to it, including connecting, refining, outputting, displaying, adjusting, generating, and any other suitable performance. Processing of the code 1130-1136 may cause the device 1100 to perform the operations 600 of FIG. 6, and any aspects related to them, including connecting, refining, outputting, displaying, adjusting, generating, and any other suitable performance.

The one or more processors 1110 include circuitry configured to implement (e.g., execute) the code stored in the computer-readable medium/memory 1112, including circuitry 1118-1124 for performing techniques described herein, according to aspects of the present disclosure. Processing circuitry 1118-1124 may cause the device 1100 to perform the method 1000 described with respect to FIG. 10, or any aspect related to it, including connecting, refining, outputting, displaying, adjusting, generating, and any other suitable performance. Processing with circuitry 1118-1124 may cause the device 1100 to perform the operations 600 of FIG. 6, and any aspects related to them, including connecting, refining, outputting, displaying, adjusting, generating, and any other suitable performance.

The device 1100 may optionally include a GPU processing system 1106. The GPU processing system 1106 may be configured to perform processing functions for the device 1100.

The optional GPU processing system 1106 includes one or more processors 1114. The one or more processors 1114 are coupled to a computer-readable medium/memory 1116 via a bus. The one or more processors 1114 and the computer-readable medium/memory 1116 may optionally communicate with the one or more processors 1110 and the computer-readable medium/memory 1112 of the CPU processing system 1104 via an MPI 1108. In certain aspects, the computer-readable medium/memory 1116 is configured to store instructions (e.g., computer-executable code) that when executed by the one or more processors 1114, cause the one or more processors 1114 to perform the method 1000 described with respect to FIG. 10, or any aspect related to it, and/or the operations 600 of FIG. 6, and any aspects related to them. Note that reference to a processor performing a function of device 1100 may include one or more processors performing that function of device 1100.

In the depicted example, computer-readable medium/memory 1116 stores code 1150-1156 (e.g., executable instructions) for performing certain functions according to aspects of the present disclosure. Processing of the code 1150-1156 may cause the device 1100 to perform the method 1000 described with respect to FIG. 10, or any aspect related to it, including connecting, refining, outputting, displaying, adjusting, generating, and any other suitable performance. Processing of the code 1150-1156 may cause the device 1100 to perform the operations 600 of FIG. 6, and any aspects related to them, including connecting, refining, outputting, displaying, adjusting, generating, and any other suitable performance.

The one or more processors 1114 include circuitry configured to implement (e.g., execute) the code stored in the computer-readable medium/memory 1116, including circuitry 1142-1148 for performing certain functions according to aspects of the present disclosure. Processing with circuitry 1142-1148 may cause the device 1100 to perform the method 1000 described with respect to FIG. 10, or any aspect related to it, including connecting, refining, outputting, displaying, adjusting, generating, and any other suitable performance. Processing with circuitry 1142-1148 may cause the device 1100 to perform the operations 600 of FIG. 6, and any aspects related to them, including connecting, refining, outputting, displaying, adjusting, generating, and any other suitable performance.

Various components of the device 1100 may provide means for performing the method 1000 described with respect to FIG. 10, or any aspect related to it, and/or the operations 600 of FIG. 6, and any aspects related to them.

### Example Aspects

Implementation examples are described in the following numbered aspects:

Aspect 1: A method performed by an apparatus, comprising: connecting a first medical data repository to a first node, the first node configured to obtain a set of medical data from the first medical data repository and refine the set of the medical data; refining, based on one or more parameters, the set of medical data to produce a set of refined medical data; and outputting the set of the refined medical data, the set of the refined medical data configured for seamless integration with a second medical data repository.

Aspect 2: The method of aspect 1, further comprising connecting the first node to a second node, the second node configured to obtain the set of the medical data from the first node and refine the set of the medical data.

Aspect 3: The method of aspect 2, wherein refining the set of the medical data is at the first node, at the second node, or at both the first node and the second node.

Aspect 4: The method of any one of aspects 2 and 3, wherein connecting the first node to the second node is performed by a user.

Aspect 5: The method of any one of aspects 2 through 4, wherein the second node comprises at least one of a reasoning node, a programming node, a visualization node, a text node, a data retrieval node, an order node, a task node, an artificial intelligence (AI) node, and note bar.

Aspect 6: The method of aspect 5, wherein the first node comprises at least one of a second reasoning node, a second programming node, a second visualization node, a second text node, a second data retrieval node, a second order node, a second task node, and a second AI node, and the first node may be the same or different from the second node.

Aspect 7: The method of any one of aspects 1 through 6, further comprising displaying a visualization on a user interface (UI) based on the set of the refined medical data.

Aspect 8: The method of any one of aspects 1 through 7, wherein the first node is an artificial intelligence (AI) node.

Aspect 9: The method of aspect 8, wherein the AI node may provide, to a user, a selection panel, an AI properties panel, or both the selection panel and the AI properties.

Aspect 10: The method of any one of aspects 8 and 9, wherein refining the set of the medical data comprises applying an inference model to the set of the medical data at the AI node.

Aspect 11: The method of aspect 10, wherein the inference model is pre-selected within the AI node.

Aspect 12: The method of any one of aspects 1 through 11, wherein the second medical data repository comprises at least one of an electronic health record (EHR) system, an electronic medical record (EMR) system, and a medical billing system.

Aspect 13: The method of any one of aspects 1 through 12, wherein the second medical data repository comprises a second node or a note bar.

Aspect 14: The method of any one of aspects 1 through 13, wherein the one or more parameters are selected by a user.

Aspect 15: The method of any one of aspects 1 through 14, wherein the one or more parameters comprise at least one of: a data type, a data filter, a data source, conditional logic, programming logic, an arithmetic operator, a comparison operator, a custom text input, a text formatting, an input connector, an output connector, a data visualization format, an artificial intelligence (AI) model, parameters to adjust performance the AI model, and any combination herein.

Aspect 16: The method of any one of aspects 1 through 15, wherein the first node comprises at least one of a reasoning node, a programming node, a visualization node, a text node, a data retrieval node, an order node, a task node, and note bar.

Aspect 17: The method of any one of aspects 1 through 16, wherein at least one of the set of the medical data and the set of the refined medical data comprise at least one of: numeric data, sequence type data, Boolean data, set data, dictionary data, binary type data, text data, date-and-time data, demographic data, vital signs data, medical orders data, lab results data, imaging studies data, and clinical notes data.

Aspect 18: The method of any one of aspects 1 through 17, wherein the first medical data repository comprises a database provided to the apparatus by a user, a database external to the apparatus, or a database internal to the apparatus.

Aspect 19: The method of any one of aspects 1 through 18, wherein the apparatus is configured to communicate with a system, the system associated with the first medical data repository, the second medical data repository, or both the first medical data repository and the second medical data repository.

Aspect 20: The method of any one of aspects 1 through 19, wherein the apparatus is configured to communicate via at least one of wired communication, wireless communication, peer-to-peer (P2P) communication, and client-server communication.

Aspect 21: An apparatus, comprising: a memory comprising executable instructions; and a processor configured to execute the executable instructions and cause the apparatus to perform a method in accordance with any one of Aspects 1-20.

Aspect 22: An apparatus, comprising means for performing a method in accordance with any one of Aspects 1-20.

Aspect 23: A non-transitory computer-readable medium comprising executable instructions that, when executed by a processor of an apparatus, cause the apparatus to perform a method in accordance with any one of Aspects 1-20.

Aspect 24: A computer program product embodied on a computer-readable storage medium comprising code for performing a method in accordance with any one of Aspects 1-20.

### Additional Considerations

The preceding description is provided to enable any person skilled in the art to practice the various aspects described herein. The examples discussed herein are not limiting of the scope, applicability, or aspects set forth in the claims. Various modifications to these aspects will be readily apparent to those skilled in the art, and the general principles defined herein may be applied to other aspects. For example, changes may be made in the function and arrangement of elements discussed without departing from the scope of the disclosure. Various examples may omit, substitute, or add various procedures or components as appropriate. For instance, the methods described may be performed in an order different from that described, and various actions may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). The singular forms “a,” “an,” and “the” include plural referents, unless the context clearly dictates otherwise. Within a claim, reference to an element in the singular is not intended to mean “one and only one” unless specifically so stated, but rather “one or more.” Unless specifically stated otherwise, the term “some” refers to one or more.

As used herein, the term “determining” encompasses a wide variety of actions. For example, “determining” may include calculating, computing, processing, deriving, investigating, updating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, simulating, choosing, establishing, and the like.

The methods disclosed herein comprise one or more operations or actions for achieving the methods. The method operations and/or actions may be interchanged with one another without departing from the scope of the claims. In other words, unless a specific order of operations or actions is specified, the order and/or use of specific operations and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in the figures, those operations may have corresponding counterpart means-plus-function components with similar numbering.

When the word “approximately” or “about” are used, this term may mean that there may be a variance in value of up to +10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

As used, terms such as “first” and “second” are arbitrarily assigned and are merely intended to differentiate between two or more components of a system, an apparatus, or a composition. It is to be understood that the words “first” and “second” serve no other purpose and are not part of the name or description of the component, nor do they necessarily define a relative location or position of the component. Furthermore, it is to be understood that that the mere use of the term “first” and “second” does not require that there be any “third” component, although that possibility is envisioned under the scope of the various embodiments described.

The following claims are not intended to be limited to the aspects shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

Unless defined otherwise, all technical and scientific terms used have the same meaning as commonly understood by one of ordinary skill in the art to which these systems, apparatuses, methods, processes and compositions belong.

The following claims are not intended to be limited to the embodiments provided but rather are to be accorded the full scope consistent with the language of the claims.

What is claimed is:

1. A method for constructing a medical informatics workflow, comprising:
connecting a first medical data repository to a data retrieval node, the data retrieval node configured to obtain a first medical data set from the first medical data repository;
transferring a first medical data set to the data retrieval node from the first medical data repository;
configuring the data retrieval node to refine the first medical data set using one or more data retrieval parameters;
refining, based on the one or more data retrieval parameters, the first medical data set to produce a first refined medical data set at the data retrieval node;
transferring the first refined medical data set to a programing node from the data retrieval node;
configuring an AI node to refine an AI data set using one or more AI parameters;
refining, based on the one or more AI parameters, the AI data set to produce a refined AI data set;
transferring the refined AI data set to the programming node from the AI node;
processing the first refined medical data set, wherein the programming node applies the refined AI data to process the first refined medical data set to generate a first processed medical data set; and
outputting the first processed medical data set to a second medical data repository; and
integrating the first processed medical data set with a second medical data repository.

2. The method of claim 1, further comprising:
providing the first refined medical data set from the data retrieval node to one or more additional nodes, the one or more additional nodes configured to obtain the first refined medical data set from the data retrieval node;
configuring the one or more additional nodes to refine the first medical data set using one or more additional refining parameters;
refining, based on the one or more additional refining parameters, the first refined medical data set to produce a second refined medical data set; and
processing the second refined medical data set to produce the first processed medical data set, wherein the programming node applies the refined AI data to process the second refined medical data set.

3. The method of claim 2, wherein the one or more additional nodes include a reasoning node, a programming node, a visualization node, a text node, a data retrieval node, an order node, a task node, or a note bar.

4. The method of claim 3, wherein refining, based on the one or more additional refining parameters, the first refined medical data set to produce a second refined medical data set is performed with the one or more additional nodes in sequence.

5. The method of claim 3, wherein refining, based on the one or more additional refining parameters, the first refined medical data set to produce a second refined medical data set is performed with the one or more additional nodes in parallel.

6. The method of claim 3, further comprising displaying a visualization on a user interface (UI) based on the set of the first processed medical data set.

7. The method of claim 1, wherein the one or more AI parameters include a summarizer, an information identifier, an information extractor, a drafter, an inconsistency detector, or an editor.

8. A system for constructing a medical informatics workflow, comprising;
a user interface (UI);
a medical data repository;
a workflow platform, the workflow platform comprising:
one or more nodes, wherein the one or more nodes are configured to:
refine, based on one or more data retrieval parameters, the first medical data set to produce a first refined medical data set at a data retrieval node of the one or more nodes;
transfer the first refined medical data set to a programing node of the one or more nodes from the data retrieval node;
refine, based on one or more AI parameters, an AI data set to produce a refined AI data set at an AI node of the one or more nodes;
transfer the refined AI data set to the programming node from the AI node;
process the first refined medical data set to produce a first processed medical data set, wherein the programming node applies the refined AI data to process the first refined medical data set; and
output the first processed medical data set to the UI; and
integrate the first processed medical data set with the UI;
a UI connection connecting the UI to the workflow platform to output the first refined medical data set to the UI; and
a medical data repository connection connecting the workflow platform to the medical data repository.

9. The system of claim 8, wherein the AI node may provide, to a user, a selection panel, an AI properties panel, or both the selection panel and the AI properties panel.

10. The system of claim 8, wherein refining the first medical data set comprises applying an inference model to the first medical data set at the AI node.

11. The system of claim 10, wherein the inference model is pre-selected within the AI node.

12. The system of claim 8, wherein the medical data repository comprises at least one of an electronic health record (EHR) system, an electronic medical record (EMR) system, and a medical billing system.

13. The system of claim 8, wherein the one or more nodes include a reasoning node, a programming node, a visualization node, a text node, a data retrieval node, an order node, a task node, or a note bar.

14. The system of claim 8, wherein the one or more nodes are further configured to display a visualization on the UI based on the first processed medical data set.

15. A non-transitory computer readable medium for constructing a medical informatics workflow, comprising computer-executable instructions that, when executed by one or more processors, cause one or more processing units to:
transfer a first medical data set to a data retrieval node of one or more nodes from a first medical data repository, wherein the first medical data repository is connected to the data retrieval node and the data retrieval node is configured to obtain the first medical data set from the first medical data repository;
configure the data retrieval node to refine the first medical data set using one or more data retrieval parameters;
refine, based on the one or more data retrieval parameters, the first medical data set to produce a first refined medical data set at the data retrieval node;
transfer the first refined medical data set to a programing node of the one or more nodes from the data retrieval node;
configuring an AI node of the one or more nodes to refine an AI data set using one or more AI parameters;
refine, based on the one or more AI parameters, the AI data set to produce a refined AI data set;
transfer the refined AI data set to the programming node from the AI node;
process the first refined medical data set, wherein the programming node applies the refined AI data to process the first refined medical data set to generate a first processed medical data set; and
output the first processed medical data set to a second medical data repository; and
integrate the first processed medical data set with a second medical data repository.

16. The non-transitory computer readable medium of claim 15, further comprising:
providing the first refined medical data set from the data retrieval node to one or more additional nodes, the one or more additional nodes configured to obtain the first refined medical data set from the data retrieval node;
configuring the one or more additional nodes to refine the first medical data set using one or more additional refining parameters;
refining, based on the one or more additional refining parameters, the first refined medical data set to produce a second refined medical data set; and processing the second refined medical data set to produce the first processed medical data set, wherein the programming node applies the refined AI data to process the second refined medical data set.

17. The non-transitory computer readable medium of claim 15, wherein the one or more nodes comprises at least one of a reasoning node, a programming node, a visualization node, a text node, a data retrieval node, an order node, a task node, and note bar.

18. The non-transitory computer readable medium of claim 15, wherein at least one of the first medical data set and the first refined medical data set comprise at least one of: numeric data, sequence type data, Boolean data, set data, dictionary data, binary type data, text data, date-and-time data, demographic data, vital signs data, medical orders data, lab results data, imaging studies data, and clinical notes data.

19. The non-transitory computer readable medium of claim 15, wherein the first medical data repository comprises a database provided by a user, an external database, or an internal database.

20. The non-transitory computer readable medium of claim 15, wherein the one or more data retrieval parameters comprise at least one of: a data type, a data filter, a data source, conditional logic, programming logic, an arithmetic operator, a comparison operator, a custom text input, a text formatting, an input connector, an output connector, a data visualization format.
